(19) United States
(12) Patent Application Publication
(10) Pub. No.: US 2005/0288636 A1
(43) Pub. Date: Dec. 29, 2005

(54) Needle removal device

(76) Inventors: Stephen Francis Gerald Cooley, Banbury (GB); Philip William Field, Banbury (GB)

Correspondence Address: PAUL E. MILLIKEN, 9061 WALL STREET, NW, MASSILLON, OH, 44646-1676, US

(21) Appl. No.: 11/153,779

(22) Filed: Jun. 15, 2005

(30) Foreign Application Priority Data
Jun. 19, 2004 (GB) 0413759.2

(51) Int. Cl. A61M 1/00; A61M 5/178; A61M 5/00

(52) U.S. Cl. 604/187; 604/27; 604/36; 604/190

(57) ABSTRACT

Apparatus 10 for the removal of a needle N from a syringe S and comprising a housing 13 having an opening 119 for insertion of a needle assembly A, a needle receiving socket 47 aligned with the opening 119. The socket 47 is formed from a pair of jaws 47 and having a central cavity with an internal profile 54 shaped to receive a needle hub H and accommodate the needle N, the jaws 47 move together to grip the needle hub H and move apart to eject the unscrewed needle assembly A, the whole socket 47 being rotatable to unscrew the needle assembly.

### FIELD OF THE INVENTION

This invention is a means of safely removing used needles from the body of a multi-use syringe and single use syringe without the need to undo and remove the needle manually

### BACKGROUND OF THE INVENTION

Multi-use and single use syringes are used in medicine, veterinary practice and in dentistry. In dentistry multi-use syringes are used for the administration of anaesthetic injections into the mouth, or may be used for mass-vaccination programs. Devices are known for the removal of used disposable needles from the syringe. Typically a needle disposal container may be provided with a lid having a shaped aperture for engagement with a needle hub. The technician or nurse carefully inserts the needle into the aperture so that the needle hub is held stationary within the aperture and then unscrews the syringe from the needle hub and the needle drops into the disposal container.

In another known apparatus shown in FR 2609401, there is disclosed a disposal container fitted with a needle gripping device that facilitates needle removal. EP-A-0838 228 discloses a motorised needle removal device which uses serrated arms for engaging the needle hub for unscrewing the needle. The needles then fall freely into the container after removal. However, if the frictional engagement is insufficient to unscrew the needle hub then the needle will remain in place. An alternative unscrewing action is shown in U.S. Pat. No. 5,531,323 in which a rotatable gear wheel engages the external surface of the needle hub.

The applicants earlier patent application GB-A-2376 892 describes apparatus for the removal of a needle assembly having a needle hub which is screw-threadably engaged with a syringe, said apparatus comprising a housing having an opening for insertion of the needle assembly and adjacent portion of the syringe, a socket mounted within the body in alignment with the opening, said socket having a cavity internally shaped to receive the hub and a coaxial through-bore to accommodate the needle, the socket holding the hub rotationally fast relative thereto allowing the syringe to be unscrewed from the needle assembly, and an ejector means located in the housing for ejecting the unscrewed needle assembly from the socket into a collection container located within the body.

The present invention provides an automatic needle removal device in which the needle is removed from the syringe body by rotation of the needle hub and is collected in a collection bin and which is a development of the earlier apparatus described above.

### STATEMENTS OF THE INVENTION

According to the present invention there is provided apparatus for the removal of a needle assembly from the body of a syringe, said apparatus comprising a housing having an opening for insertion of the needle assembly and adjacent portion of the syringe, a needle receiving socket mounted within the body in alignment with the opening, said socket having a cavity internally shaped to receive the needle hub and accommodate the needle, the socket holding the hub rotationally fast relative thereto and being rotatable relative to the syringe to remove the needle assembly from the syringe, wherein the socket is formed from at least two jaws which are moved together to grip the needle hub to facilitate unscrewing of the needle hub and which are moved apart to eject the removed needle assembly into a collection bin.

The term syringe covers both syringes with disposable plastic bodies typically having push on needle hubs and multi-use re-usable syringes, typically made of metal and/or glass and having screw on needle hubs.

Preferably the socket comprises two semi-circular jaws which are rotated by cam means which close the jaws in one direction of rotation relative to the socket to remove the needle assembly and which open the jaws in the reverse direction of rotation. The two jaws may also be biased apart by spring means.

Preferably the jaws have an internal profile shaped to accommodate a plurality of different sized hubs.

The jaws may be located in a housing which is rotated by a motor, the housing being capable of limited rotational movement relative to the jaws and including cam surfaces which act on the jaws during said limited rotational movement to open and close the jaws.

The motor is a suitable electrical bi-directional motor which rotates the housing through a gear train clamp the hub and unscrew the hub from the needle body in one direction and release the hub in the other direction. The motor is preferably a 12V motor torque sensing motor which may be operated from the mains via a transformer or may be battery operated, preferably using rechargeable batteries.

Preferably, the housing has a gear formed integrally therewith.

The motor may be activated by a manual switch means located inside of the opening and which is operated by insertion of the syringe body into said opening. A second manual switch may be located externally of the housing. The apparatus housing in use sits directly on the collection bin, the bin having an aperture for receiving the needle assemblies, and which is provided with a rotatable closure which also serves as the second switch for the motor, the motor being activated when the closure is in an open condition. The motor is operable only when both the switches are closed.

The housing may connected to the collection bin by a locking means which is unlocked to allow the bin to be removed from the housing for disposal of needle assemblies. Additionally or alternatively, the housing may be connected a resilient clip means on one of the housing and bin which engages a detent on the other of the housing and bin.

The locking means may also be provided by the closure which when open couples the housing to the bin and closed releases the housing from the collection bin for detachment therefrom.

The apparatus housing includes a motor support plate which is shaped to locate the motor, the shaped portion of the support plate being received in a like recess formed in the collection bin to facilitate location of the apparatus on the location bin.

The collection bin may be provided as a part of said apparatus according to the present invention.

The bin may be made from a suitable plastic material to enable both bin and contents to be incinerated, and for withstanding elevated temperatures used for cleaning

### DESCRIPTION OF THE DRAWINGS

The invention will be described by way of example and with reference to the accompanying drawings in which:

FIG. 1 is an isometric view of apparatus according to the present invention also showing the insertion of a syringe and needle;

FIG. 2 is an exploded isometric view of the apparatus of FIG. 1;

FIG. 3 is a plan view of the apparatus of FIG. 1 with the housing removed to show the drive mechanism for the needle receiving socket;

FIG. 4 is a section on the line 4-4 of FIG. 3;

FIG. 5 is a section on the line 5-5 of FIG. 3;

FIG. 6 is a side elevation of the apparatus with the housing removed to show the internal workings; and

FIG. 7 is an exploded isometric view showing the needle removal assembly in an assembled condition.

### DETAILED DESCRIPTION OF THE INVENTION

With reference to FIG. 1, there is shown a typical dental syringe S, having a needle assembly A, screwed onto the body B of the syringe. The needle assembly comprises needle N which is mounted within a hub H which screws onto the body B. Disposable needle assemblies A are removable from the body by gripping or holding the external surface of the hub H and unscrewing.

With reference also to FIG. 7, the needle assembly A is removed from the re-usable syringe S using the needle remover apparatus 10. The syringe S may be used for medical, dental or veterinary purposes. The apparatus 10 is formed in two distinct parts, a needle remover 11 and a collection bin 12 on which the needle remover 11 sits. The needle remover 11 comprises a hollow housing 13 and a sub-assembly 14.

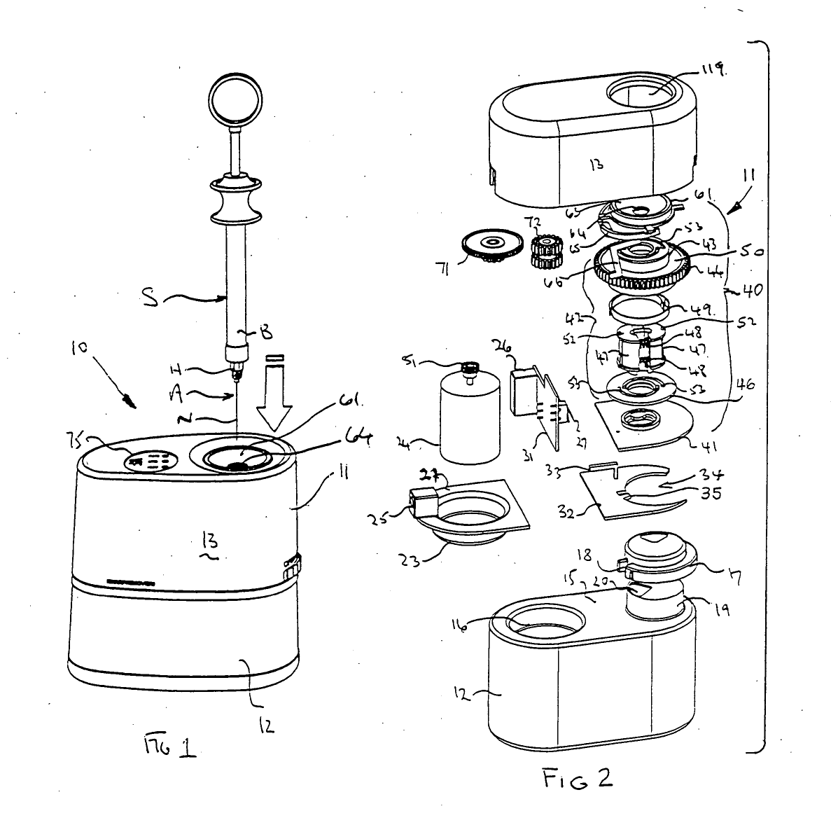

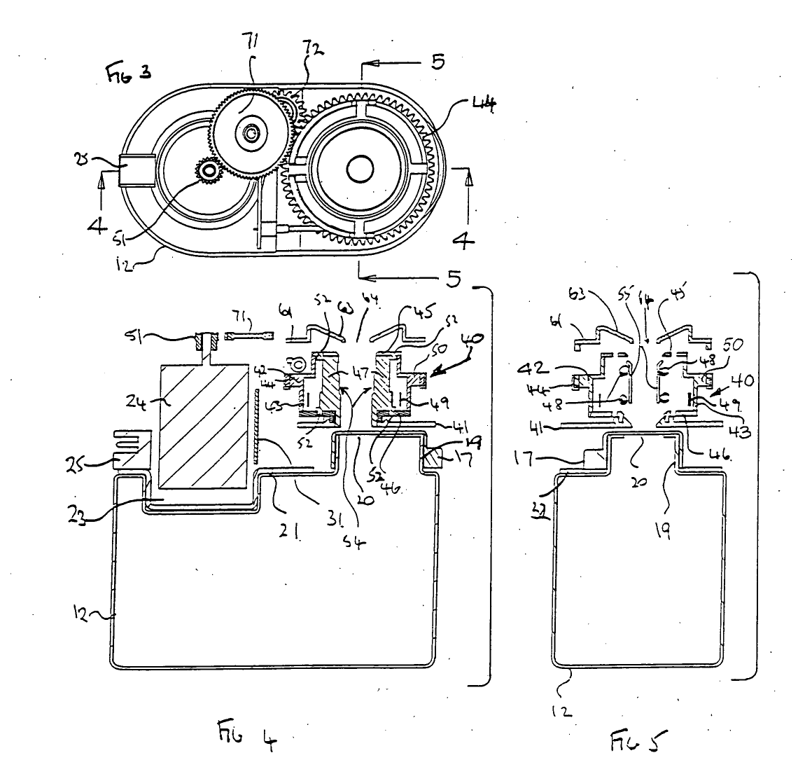

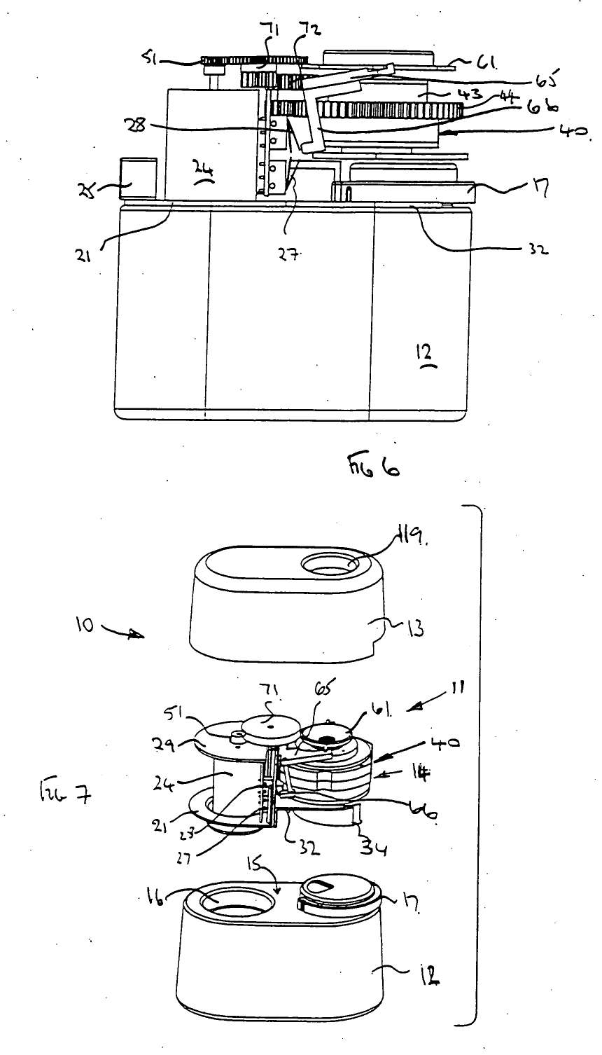

Referring now to FIGS. 2-6 also, the sub assembly 14 comprises a base plate 21 which has a recess 23 therein and supports an upright PCB (printed circuit board) 31 on its inner side. The recess 23 in use locates in a like recess 16 in the top surface 15 of the bin 12 and houses an electric motor 24 in an upright condition. Also located on the base plate 21 is a electrical power socket 25 for a power connection to the PCB 31. The PCB 31 includes an AC/DC transformer 26, electrical contacts 27, 28, and programmable microprocessor (not shown) for control of the motor 24. The motor 24 is held in its upright position and has a shaft with a drive gear 51 attached thereto.

A switch plate 32 sits on the upper surface 15 of the bin 12 to the side of the base plate 21 and includes an arcuate clip portion 34 which in use clips around a raised circular boss 19 on the upper surface 15 of the bin 12. The boss 19 has an aperture 20 therein and has a closure 17 mounted on the boss for opening and closing the aperture 20. The switch plate 32 includes a leg 33 which is engageable with a contact 27 for operation of the motor 24 and a tab 35 which in use engages a recess 18 in the outer surface of the closure 17 such that rotation of the closure 17 on the boss 19 causes the lug 33 to open or close the contact 27.

A gripping jaw assembly 40 for gripping the needle hub H is mounted above the closure 17. The assembly 40 includes a base plate 41 on which a housing 42 for the jaws is rotatably mounted. The base plate 41 in use clips into the housing 42. The jaw housing 42 comprises a cylindrical hollow body 43 having a coaxial outer flange 50 with a gear 44 on its radially outer surface, and annular cam plates 45,46 at its upper and lower ends. A pair of arcuate jaws 47 are located within coaxially within the cylindrical housing 42. The two jaws 47 are biased apart by four springs 48 and may additionally also be biased apart by a spiral spring 49. The upper and lower ends of the two jaws 47 are provided with lugs or pins 52 which engage cam surfaces provided in slots 53 (see FIG. 2) in the respective cam plates 45 46. Rotation of the housing 42 relative to the jaws 47 causes the lugs 52 to move along the slots 53 simultaneously moving the jaws 47 radially relative to the axis of rotation of the housing 42.

The two jaws 47 each have a guide plate 55 thereon to assist and guide movement of the jaws 47 towards and way from each other. The jaws 47 form a generally cylindrical socket located within the housing 13 in alignment with an opening 119 in the top surface of the housing 13. The two jaws 47 form a split open ended socket having a internal stepped profile 54 (see FIG. 4) which can accept a plurality of different sized needle hubs H. The stepped profile 54 forms a split cavity with the larger diameter portion open to the opening 119 and the smallest diameter portion at the base of the cavity.

An annular activation plate 61 is slideably mounted on the flange 50 on the housing 42 by means of pins (not shown). The plate 61 is capable of limited axial movement relative to the housing 42 and is biased away from the flange 50 by springs arranged coaxially of the pins. The movement away from the flange 50 may be limited by abutments on the pins. The activation plate 61 fills the opening 119 in the housing 13 and has a downwardly extending conical portion 63 with an aperture 64 in the centre which aligns with the cavity between the jaws 47 and the aperture 119 in the housing 13. A semi-circular switch means 65 is pivotally mounted to the activation plate 61 and has a dog leg shaped limb 66 thereon which in use activates contact 28 on the PCB 31 causing the motor 24 to operate.

The housing 42 is caused to rotate by operation of the motor 24. The motor 24 is preferably a bi-directional motor which is torque sensitive delivering a suitable maximum torque load removal of a needle N. The operation of the motor is controlled by the programmed controller on the PCB 31. The gear wheel 51 on the motor is connected to the gear 44 on the housing 42 by gears 71, 72, the gear 71 being a reduction gear.

The PCB 31 may be connected to an LED display 75 on the top of the housing 13 which indicates various stages of operation of the needle remover apparatus 10.

In use, the apparatus 10 is first activated by rotation of the closure switch 17 which opens the aperture 20 and causes the leg 33 to close contact 27 on the PCB. This may illuminate a warning LED on the display 75.

A needle N is inserted through the opening 119 in the housing 13 and aperture 64 until the needle body B engages the activation plate 61. Further inward movement of the needle body B depresses the activation plate 61 locating the needle hub H between the jaws 47 and causing the switch means 65 to move the limb 66 to operate contact 28 on the PCB. The needle N passes through the gap between the jaws 47.

The closure of contact 28 operates the motor 24 which via gears 51,71,72 rotates the jaw housing 42 in one direction. The jaws 47 are connected to the housing 42 by the lugs 52 engaging in the slots 53 in the cam plates 45,46. Limited rotation of the housing 42 relative to the jaws 47 moves the lugs along the slots 53 moving the jaws together to clamp the hub H. Once the lugs 52 abut the end of the respective slot 53, further rotation of the housing 42 unscrews the needle and hub from the syringe body. The motor 24 is programmed to rotate the housing through an appropriate number of rotations over about a 3 sec. time period.

After the programmed rotation in one direction, the microprocessor operates a warning LED on the display 75 and the syringe S is then removed. The motor 24 operates in the reverse direction opening the jaws 47. On opening of the jaws, the needle N will eject from the jaws and drop into the collection bin 12.

The bin 12 can be removed from the apparatus when full. The closure 17 closes the aperture 20 and the bin and its contents can be disposed of by any suitable method e.g incineration.

The assembly 40 comprising the jaw housing 42, activation plate 61 and switch means 65 can be removed from the housing 13 as a unit for cleaning.

The apparatus may also be used with disposable syringes having needles whose needle hub is a push fit onto the syringe body.

1. Apparatus for the removal from a syringe of a needle being part of a needle assembly having a needle hub attached to the body of the syringe, said apparatus comprising a housing having an opening for insertion of the needle assembly and adjacent portion of the syringe, a needle receiving socket mounted within the body in alignment with the opening, said socket having a cavity internally shaped to receive the needle hub and accommodate the needle, the socket holding the hub rotationally fast relative thereto and being rotatable relative to the syringe to remove the needle assembly from the syringe, wherein the socket is formed from at least two jaws which are moved together to grip the needle hub to facilitate rotation of the needle hub and which are moved apart to eject the removed needle assembly into a collection bin.

2. Apparatus as claimed in claim 1 wherein the socket comprises two semi-circular jaws which are rotated by cam means which close the jaws in one direction of rotation and which open the jaws in the other direction of rotation.

3. Apparatus as claimed in claim 2, wherein the two jaws are also biased apart by spring means.

4. Apparatus as claimed in claim 1, wherein the semi-circular jaws have an internal profile enabling the jaws to be utilised with a plurality of different needle hubs.

5. Apparatus as claimed in claim 1, wherein the jaws are located in a housing which is rotated by a motor, the housing being capable of limited rotational movement relative to the jaws and including cam surfaces which act on the jaws during said limited rotational movement to open and close the jaws.

6. Apparatus as claimed in claim 5 wherein the motor is an electrical bi-directional motor which rotates the housing via gear train.

7. Apparatus as Claimed in claim 5, wherein the housing has a gear formed integrally therewith on its outer surface.

8. Apparatus as claimed in claim 5, wherein the motor is activated by a manual switch means located inside of the opening in the housing and which is operated by insertion of the syringe body into said opening.

9. Apparatus as claimed in claim 5, wherein the housing for the jaws and the switch means are removable as a unit from the apparatus housing for sterilisation.

10. Apparatus as claimed in claim 8, wherein the apparatus housing sits directly on the collection bin, the bin having an aperture for receiving the needle assemblies and which is provided with a rotatable closure which also serves as a second switch for the motor, the motor being activated when the closure is in an open condition.

11. Apparatus as claimed in claim 10, wherein the motor is operable only when both the switches are closed.

12. Apparatus as claimed in claim 10, wherein the apparatus housing includes a motor support plate which is shaped to locate the motor, the shaped portion of the support plate being received in a like recess formed in the collection bin to facilitate location of the apparatus on the location bin.

13. Apparatus as claimed in claims 1 and further including the collection bin.

14. Apparatus for the removal from a syringe of a needle assembly having a needle hub attached to the body of the syringe, said apparatus comprising a housing having an opening for insertion of the needle assembly and adjacent portion of the syringe, a needle receiving socket mounted within the body in alignment with the opening, said socket comprising a pair of semi-circular jaws which move together to grip the needle hub for removing said hub and which move apart to eject the unscrewed needle assembly, the jaws having an internal profile enabling the jaws to be utilised with a plurality of different needle hubs and accommodate the needle, the socket holding the hub rotationally fast relative thereto and being rotatable relative to the syringe to remove the needle assembly from the syringe, for ejection into a collection bin, and said jaws are located in a housing which is rotated by a motor, the housing being capable of limited rotational movement relative to the jaws and including cam surfaces which act on the jaws during said limited rotational movement to open and close the jaws.
